(19) United States
(12) Patent Application Publication
(10) Pub. No.: US 2002/0022043 A1
(43) Pub. Date: Feb. 21, 2002

(54) Method for killing house dust mites in clothing and other soft materials

(76) Inventor: Jeffrey D. Miller, Ridgefield, CT (US)

Correspondence Address: DARBY & DARBY P.C., 805 Third Avenue, New York, NY, 10022, US

(21) Appl. No.: 09/751,325

(22) Filed: Dec. 28, 2000

Related U.S. Application Data
(60) Provisional application No. 60/173,340, filed on Dec. 28, 1999.

(51) Int. Cl. A01N 25/34; A01N 65/00; A61K 35/78

(52) U.S. Cl. 424/403; 424/725

(57) ABSTRACT

The present inventor has found, surprisingly, that exposing woolen or other fabrics to the vapors of certain pleasant-smelling plant oils, including wintergreen oil, lavandin oil, Ylang-Ylang oil, and others, kills house dust mites in those fabrics. In one aspect of the present invention, a method for killing house dust mites which are living in or infesting a fabric substrate is disclosed. The method includes the step of exposing said mite-infested substrate to a vapor of at least one plant oil. The plant oil is selected from the group of Anise, Balsam, Basil, Bay, Birch, Cajeput, Camphor, Caraway, Cinnamon, Clove, Coriander, Dill, Fennell, Fir, Garlic, Lavender, Lavendin, Lemongrass, Marjoram, Nutmeg, Peppermint, Pine, Rosemary, Rue, Sage, Spearmint, Tea Tree, Thuja, Thyme, Wintergreen and Ylang-Ylang. In a preferred method, the exposing step comprises placing the mite-infested substrate in a closed drawer or other closed container along with a volume of the selected plant oil. The plant oil can be applied to the substrate in a variety of manners as can be understood from the following detailed description.

### FIELD OF THE INVENTION

This invention pertains to a method for killing house dust mites in soft materials such as clothing.

### BACKGROUND OF THE INVENTION

It has been known for many years that common house dust is an important cause of asthma, rhinitis and eczema in allergic individuals. The mite Dermatophagoides pteronyssinus and related mites are the major sources of house dust allergen in temperate climates including the United States. Dust mites are eight-legged arachnids, relatives of spiders. They live in close association with humans, their main food source being the shed scales from skin. A major protein allergen present in the fecal particles of Dermatophagoides pteronyssinus, Der p I, can cause acute asthma symptoms when present at levels greater than 10μg per gram of dust.

Dust mites are photophobic, and live within soft materials such as pillows, mattresses, blankets and clothing. One study revealed 20,000 live dust mites in a jacket. Another study showed mean Der p I levels of 15.9 μg/gm in all clothing and 21.0 μg/gm in woolen garments. Hot water washing, dry cleaning, freezing and prolonged sun exposure can kill mites in fabrics. These methods are not practical for all fabrics, or may not be practical to do frequently.

Since mites are not insects and are not even closely related to insects, it is not surprising that many insecticides do not kill mites. Chemical miticides exist which can kill some types of mites, but most of these have been developed for agricultural use, to kill plant-eating mites which infest growing crops or stored grain, and which constitute different mite species. Use of a chemical substance indoors, and particularly on clothing or carpeting in close contact with the user, obviously requires that the chemical be safe and pleasant.

Despite advances in treatment, morbidity, mortality and costs related to asthma and other allergic diseases continue to rise. Since house dust mites are a major cause of these conditions, there is a need in the art for a safe and effective way of killing mites in clothing and other fabrics.

### SUMMARY OF THE INVENTION

The present inventor has found, surprisingly, that exposing woolen or other fabrics to the vapors of certain pleasant-smelling plant oils, including wintergreen oil, lavandin oil, Ylang-Ylang oil, and others, kills house dust mites in those fabrics.

In one aspect of the present invention, a method for killing house dust mites which are living in or infesting a fabric substrate is disclosed. The method includes the step of exposing said mite-infested substrate to a vapor of at least one plant oil. The plant oil may be selected from the group of Anise, Balsam, Basil, Bay, Birch, Cajeput, Camphor, Caraway, Cinnamon, Clove, Coriander, Dill, Fennell, Fir, Garlic, Lavender, Lavendin, Lemongrass, Marjoram, Nutmeg, Peppermint, Pine, Rosemary, Rue, Sage, Spearmint, Tea Tree, Thuja, Thyme, Wintergreen and Ylang-Ylang.

In a preferred method, the exposing step comprises placing the mite-infested substrate in a closed drawer or other closed container along with a volume of the selected plant oil. The plant oil can be applied to the substrate in a variety of manners as can be understood from the following detailed description.

These and other features and aspects of the present invention can be better appreciated from the following description of certain preferred embodiments.

### DETAILED DESCRIPTION OF CERTAIN PREFERRED EMBODIMENTS

I discovered that exposing woolen clothing or other soft fabrics infested with house dust mites to the vapors of several plant oils—including the oils of Anise, Balsam, Basil, Bay, Birch, Cajeput, Camphor, Caraway, Cinnamon, Clove, Coriander, Dill, Fennell, Fir, Garlic, Lavender, Lavendin, Lemongrass, Marjoram, Nutmeg, Peppermint, Pine, Rosemary, Rue, Sage, Spearmint, Tea Tree, Thuja, Thyme, Wintergreen and Ylang-Ylang—kills the mites in such clothing as well as their eggs.

The oils used are aromatherapy-grade oils produced by steam distillation of the specific plant, and readily available commercially from many suppliers. These oils release vapors spontaneously, and thus can be placed in contact with the mite infested substrate in many ways, e.g. placing a few drops of the oil on an absorbent material such as a paper towel or piece of cotton, which is then placed in a closed container, such as a closed drawer or a sweater storage box, containing the infested fabric. Alternatively, several drops of the oil may be placed in a plastic or glass dish within such a closed container. Application may also be effected by spraying a mist of the oil onto the substrate by means of a simple atomizer, and then placing the treated object into a sealed container that allows the accumulation of the oil vapor. In the case of substrates which can not be placed within closed containers for treatment, such as carpets, the vapor of the oil may be contained long enough for it to have its desired effect by placing an impermeable barrier such as plastic sheeting over the carpet which has been sprayed with the oil, and leaving such barrier in place for a sufficient time to kill mites, such as, for example, 24 hours. Additional methods of dispersing the vapor of the oils include the use of a packet containing a solution or gel of the plant oil having on at least one surface of the packet a permeable membrane through which the vapor volatilizes either spontaneously or with the help of electric heat, as in an air freshener. All of these methods have proven to be effective.

The substrates encompassed by the methods of the present invention include without limitation clothing, carpeting, bedding materials, upholstery, toys made from fabrics, and other soft fabrics.

The present invention is further described in the following working examples, which are intended to illustrate the invention without limiting its scope.

### EXAMPLE 1

The ability of various plant oils to kill house dust mites was assessed by placing three drops of the oil being tested into a Petri dish containing D. pteronyssinus cultures. Cultures were examined microscopically 3, 8, 24 and 48 hours later. It was discovered that the vapor of some oils killed all mites by 8 hours, while others had no effect even after 24 hours of exposure. The following table lists those oils found to be capable of killing all of the dust mites (+), those which killed some but not all (+/−), and those which did not kill them (−).

Anise +

Atlas Cedarwood +/−

Balsam +

Basil +

Bay 1+

Bergamot +/−

Birch +

Cajeput +

Camphor +

Caraway +

Cardomom +/−

Cedar −

Cilantro −

Cinnamon +

Clove +

Coriander +

Cypress −

Dill +

Elemi +/−

Erigeron +/−

Eucalyptus +/−

Fennell +

Fir +

Garlic +

Juniperberry +/−

Lavender +

Lavendin +

Lemon +/−

Lemongrass +

Marjoram +

Nutmeg +

Orange +/−

Oregano +/−

Palmarosa −

Peppermint +

Pine +

Rosemary +

Rue +

Sage +

Spearmint +

Tea tree +

Thuja +

Thyme +

Wintergreen +

Ylang-Ylang +

### EXAMPLE 2

A wool sweater was cut into sections, which were inoculated with dense cultures of D. pteronyssinus mites. These sections, and open Petri dishes containing the mite cultures, were placed in two 15-liter plastic sweater storage boxes. Into one box was placed a 0.11 oz packet of 11.49% lavandin oil. The second was left as an untreated control.

What is claimed is:

1. A method for killing house dust mites infesting a fabric substrate, said method comprising the step of exposing said mite-infested substrate to a vapor of at least one plant oil selected from the group consisting of:
Anise, Balsam, Basil, Bay, Birch, Cajeput, Camphor, Caraway, Cinnamon, Clove, Coriander, Dill, Fennell, Fir, Garlic, Lavender, Lavendin, Lemongrass, Marjoram, Nutmeg, Peppermint, Pine, Rosemary, Rue, Sage, Spearmint, Tea Tree, Thuja, Thyme, Wintergreen and Ylang-Ylang.

2. The method as in claim 1, wherein the exposing step comprises placing the mite infested substrate in a closed container along with a volume of the selected plant oil.

3. The method as in claim 2, wherein the plant oil is saturated into a piece of cotton or a piece of a toweling.

4. The method as in claim 2, wherein the plant oil resides in an open dish within the closed container.

5. The method as in claim 1, wherein the exposing step comprises spraying the plant oil on the substrate.

6. The method as in claim 1, wherein the fabric substrate is one of a clothing, a bedding material, an upholstery, a carpeting, and a toy made from fabric.
